(19) United States
(12) Patent Application Publication
(10) Pub. No.: US 2023/0173301 A1
(43) Pub. Date: Jun. 8, 2023

(54) METHOD AND SYSTEM FOR CONCURRENT PHOTOTHERMAL ABLATION AND INTERSTITIAL PHOTODYNAMIC THERAPY

(71) Applicant: Health Research, Inc., Buffalo, NY (US)

(72) Inventors: Gal Shafirstein, Amherst, NY (US); David Bellnier, Buffalo, NY (US)

(21) Appl. No.: 18/162,995

(22) Filed: Feb. 1, 2023

Related U.S. Application Data
(62) Division of application No. 16/607,683, filed on Oct. 23, 2019.
(60) Provisional application No. 62/492,171, filed on Apr. 29, 2017.

(51) Int. Cl. A61N 5/06; G01K 7/36

(52) U.S. Cl. CPC A61N 5/062; A61N 5/0601; G01K 7/36; A61N 2005/0612; A61N 2005/0628; A61N 2005/063

(57) ABSTRACT

The present disclosure provides a method and a system for treating a tissue using photodynamic therapy (PDT). A photosensitizer is administered to the tissue and one or more optical fibers are placed in the tissue. A treatment light is applied to the tissue by way of the one or more optical fibers. A temperature of the tissue is measured during application of the treatment light, and a fluence rate of the treatment light is modified based on the temperature of the tissue. For example, the fluence rate may be modified to be lower if the temperature of the tissue is higher than a predetermined threshold.

### CROSS-REFERENCE TO RELATED APPLICATIONS

This application is a divisional of U.S. application Ser. No. 16/607,683, filed on Oct. 23, 2019, which is a National Phase of International Application No. PCT/US2018/030314, filed on Apr. 30, 2018, which claims priority to U.S. provisional Application No. 62/492,171, filed on Apr. 29, 2017, now pending, the disclosure of which is incorporated herein by reference.

### STATEMENT REGARDING FEDERALLY SPONSORED RESEARCH

This invention was made with government support under grant no. CA193610 awarded by the National Institutes of Health. The government has certain rights in the invention.

### FIELD OF THE DISCLOSURE

The present disclosure relates to photodynamic therapy.

### BACKGROUND OF THE DISCLOSURE

Photodynamic therapy (PDT), in particular interstitial photodynamic therapy (I-PDT), offers promising outcomes for patients with refractory locally advanced cancer. The use of I-PDT with porfimer sodium (Photofrin®) is approved for palliation in patients with esophageal cancer or lung cancer with airway obstruction, who are non-candidates for surgery or radiation therapy. In addition, I-PDT with porfimer sodium has been used, in compassionate care settings, to treat patients with head and neck squamous cell carcinoma. A principal clinical goal has been to shorten treatment times by administering the therapeutic light at high dose rates (i.e., 400 mW/cm) that are clinically approved by the FDA for I-PDT with porfimer sodium. However, the cure rate for I-PDT with porfimer sodium is limited.

PDT has also been viewed as beneficial when considering the relatively minor nature of adverse effects. Additionally, it has been noted that PDT provides excellent cosmetic outcomes. To date, it has been believed that during I-PDT the changes in tissue temperature do not affect the response. Physicians and researchers assumed that PDT is associated with minimal heating. The clinically approved, and used, light dose rate (400 mW/cm) for PDT or I-PDT with porfimer sodium was chosen arbitrarily, about 25 years ago. There was no systemic study to evaluate potential tissue heating during I-PDT. Several retrospective clinical studies suggest that PDT and I-PDT will result in retention of functional anatomy and other benefits. While offering improved palliative outcomes for patients with such advanced diseases, there remains a need for further improvement.

### BRIEF SUMMARY OF THE DISCLOSURE

In a first aspect, the present disclosure provides a method for treating a tissue. A photosensitizer is administered to the tissue. One or more optical fibers are placed in the tissue. For example, a portion (such as, for example, an end portion) of the one or more optical fibers are inserted in the tissue. The optical fibers may be spaced apart from one another such that a light dose may be applied to the tissue. The method includes applying a treatment light to the tissue by way of the one or more optical fibers. A temperature of the tissue is measured during application of the treatment light. The fluence rate (mW/cm²) of the treatment light is modified based on the temperature of the tissue. For example, by adjusting the light dose rate one may govern the intratumoral fluence rate within the tissue (e.g., intratumoral fluence rate). For example, the fluence rate may be modified to be lower if the temperature of the tissue is higher than a predetermined threshold. In some embodiments, the fluence rate is modified to maintain a tissue temperature between 50° C. and 65° C. In some embodiments, the fluence rate is modified to maintain a tissue temperature of substantially 60° C. In some embodiments, the fluence rate is modified to maintain a tissue temperature between 60° C. and 90° C.

In some embodiments, one or more dosimetry fibers may be placed in the tissue and configured to measure light dose (J/cm²). The optical fiber(s) and/or the dosimetry fiber(s) may be disposed within one or more light-transmitting catheters (LTCs) placed in the tissue. For example, each optical fiber may be disposed in a corresponding LTC. In another example, each of a plurality of LTCs may contain an optical fiber and a dosimetry fiber.

In another aspect, the present disclosure may be embodied as a system for treating a tissue. The system includes a light source and an optical fiber operably coupled to the light source. The optical fiber is configured to deliver a light dose of treatment light to the tissue. A temperature sensor is configured to measure a temperature of the tissue. The temperature sensor may be any suitable sensor such as, for example, a thermistor, a thermal imaging sensor, a fiber optic, a magnetic resonance thermometer (providing volumetric temperature data), or the like. In some embodiments, the temperature sensor is configured to measure temperature at a plurality of locations throughout the volume of the tissue. In some embodiments, the temperature sensor comprises a plurality of temperature sensitive catheters.

A controller, such as, for example, a programmable microprocessor, is in communication with the temperature sensor and configured to modify a fluence rate of the treatment light based on a measured temperature. In some embodiments, the controller is configured to modify the fluence rate of the treatment light to maintain a tissue temperature between 50° C. and 65° C. In some embodiments, the controller is configured to modify the fluence rate of the treatment light to maintain a tissue temperature of substantially 60° C. In some embodiments, the controller is configured to modify the fluence rate of the treatment light to maintain a tissue temperature between 60° C. and 90° C.

The system may further include a dosimetry fiber for measuring the light dose. A spectrometer may be operably coupled to the dosimetry fiber. The system may further include an LTC, and the optical fiber and/or the dosimetry fiber may be disposed in the LTC.

In some embodiments, a second optical fiber is operably coupled to the light source and configured to deliver a second light dose of treatment light to the tissue. The controller may be configured to modify a fluence rate of the second light dose based on the measured temperature. The fluence rate of the second light dose may be modified based on a temperature at a second location of the tissue.

### DESCRIPTION OF THE DRAWINGS

For a fuller understanding of the nature and objects of the disclosure, reference should be made to the following detailed description taken in conjunction with the accompanying drawings, in which:

FIG. 1 depicts a system according to an embodiment of the present disclosure;

FIG. 2 is a chart showing a method according to another embodiment of the present disclosure;

FIG. 3 shows a mouse being fitted with two optical fibers, each disposed within a light-transmitting catheter, for use in interstitial photodynamic therapy (I-PDT);

FIG. 4 is a chart showing intratumoral heating results for a light dose of 150 mW/cm, 100 J/cm with and without photosensitizer;

FIG. 5 is a chart showing intratumoral heating results for a light dose of 350 mW/cm, 100 J/cm with and without photosensitizer;

FIG. 6 is a chart showing tumor size over time in populations of mice, where a control population was untreated, a second population was treated with a light dose of 150 mW/cm, 100 J/cm (control vs. light p=<0.0001), and a third population was treated with I-PDT (control vs. I-PDT p=<0.0001; light vs. I-PDT p<0.05);

FIG. 7 is a chart showing tumor size over time in populations of mice, where a control population was untreated, a second population was treated with a light dose of 350 mW/cm, 100 J/cm (control vs. light p=<0.0001), and a third population was treated with I-PDT (control vs. I-PDT p=<0.0001; light vs. I-PDT p=0.339); and

FIG. 8 is a chart showing tumor size over time in populations of mice, where a control population was untreated, a second population was treated with a light dose of 100 mW/cm, 540 J/cm (control vs. light p=<0.0001), and a third population was treated with I-PDT concurrent with the same light dose as the second population (control vs. light p=<0.0001; light vs. I-PDT/light p=0.164).

### DETAILED DESCRIPTION OF THE DISCLOSURE

As mentioned above, a perceived benefit of photodynamic therapy has been the lack of significant changes in tissue temperature. However, the present disclosure advantageously utilizes increased temperatures induced in the tissue using the I-PDT techniques disclosed herein to enhance efficacy as compared to I-PDT without an increased temperature.

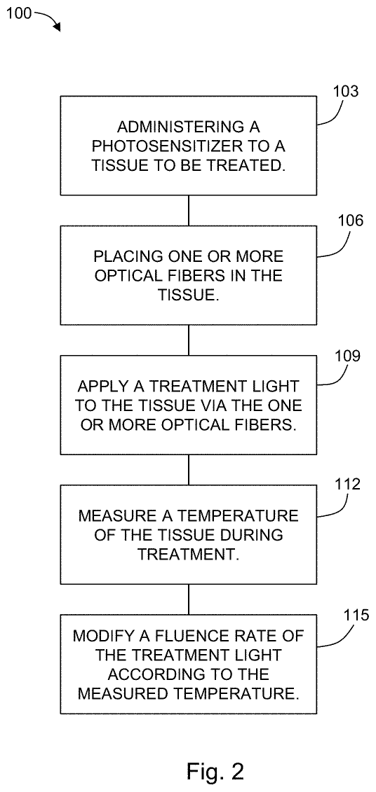

With reference to FIG. 2, the present disclosure may be embodied as a method 100 for treating a tissue, for example, treating a tumor, of an individual using interstitial photodynamic therapy (I-PDT) (see FIG. 2). The method 100 includes administering 103 a photosensitizer to the tissue. The photosensitizer may be, for example, porfimer sodium (Photofrin®) or any other photosensitizer known for use in I-PDT—e.g., capable of generating reactive oxygen species and radicals when activated by light in the presence of oxygen. The photosensitizer may be administered 103 by, for example, intravenous injection.

One or more optical fibers are placed 106 into the tissue to be treated. The optical fibers may be placed at locations in the tissue according to a predetermined treatment plan. In some embodiments, the optical fiber(s) are placed 106 into the tissue by way of light-transmitting catheter(s) (LTCs) where each optical fiber is disposed in a light-transmitting catheter.

A treatment light is applied 109 to the tissue by way of the one or more optical fibers. The treatment light has a fluence (measured in, for example, joules per square centimeter—J/cm²) and a fluence rate (measured in, for example, milliwatts per square centimeter—mW/cm²).

The method 100 includes measuring 112 a temperature of the tissue during application of the treatment light (i.e., during “treatment”). Measurement 112 of the tissue may be performed using any technique appropriate. For example, volumetric measurement may be accomplished using magnetic-resonance thermometry (MR thermometry or MRT). In another example, temperature is measured using a temperature-sensitive catheter. In embodiments where LTCs are placed in the tissue, a temperature-sensitive catheter may optionally be disposed in an LTC. Other methods for measuring 112 a temperature of a tissue may be used.

The fluence rate of the treatment light may be modified 115 based on the temperature of the tissue. The fluence rate of the treatment light within the tissue (e.g., intratumoral fluence rate) may be modified by adjusting the light dose rate. For example, the fluence rate may be decreased when the tissue temperature is higher than a (first) predetermined threshold. In this way, the temperature of the tissue will decrease. Similarly, the fluence rate of the treatment light may be increased if the tissue temperature is lower than a second predetermined threshold, which may be the same as or different from the first predetermined threshold. By increasing the fluence rate, the tissue temperature will increase. In exemplary embodiments, the fluence rate is modified to maintain a tissue temperature of between 50-65° C., inclusive. In another exemplary embodiment, the fluence rate is modified to maintain a tissue temperature of less than 60° C. In another exemplary embodiment, the fluence rate is modified to maintain a tissue temperature of substantially 60° C. By substantially, embodiments may maintain the temperature of the tissue within a desired tolerance, for example, ±5° C., ±1° C., ±0.5° C., ±0.2° C., or other tolerance levels between these exemplary values, for example, in 2° C. increments. In embodiments incorporating thermal ablation, the temperature may be maintained at greater than 60° C. and less than 100° C. (or in some embodiments, less than 90° C.) in order to avoid tissue carbonization.

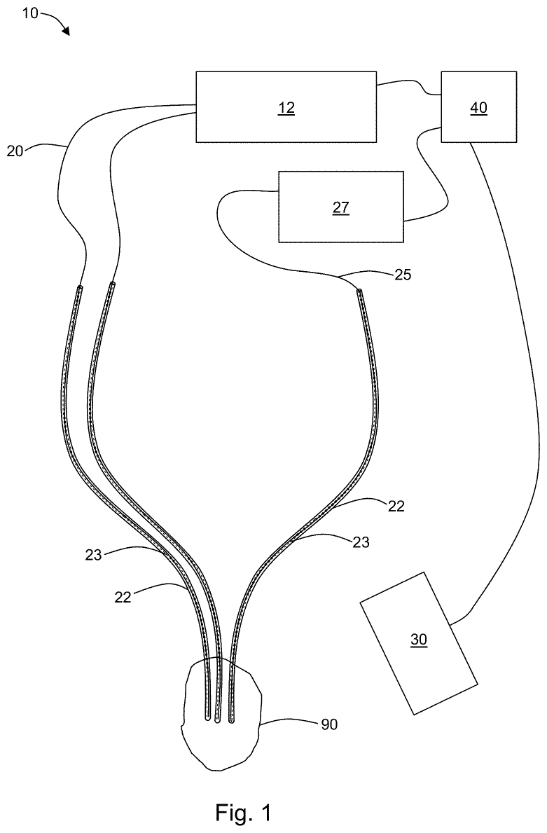

With reference to FIG. 1, the present disclosure may be embodied as a system 10 for treating tissue 90 using I-PDT is provided. The system 10 includes a light source 12. For example, the light source 12 may be a laser configured to emit light at a wavelength for activating a selected photosensitizer. For example, when using Photofrin, the emitted light may be 630 nm. An optical fiber 20 is operably coupled to the light source 12. The optical fiber 20 is configured to deliver a light dose to the tissue 90. The optical fiber 20 may have a cylindrical diffuser, a fiber with a flat-cut end, or other configuration. Embodiments of the system 10 may include additional optical fibers, for example, the embodiment depicted in FIG. 1 includes a second optical fiber 20 for delivering a second light dose to the tissue 90.

The system 10 includes a temperature sensor 30 for measuring a temperature of the tissue 90. In some embodiments, the temperature sensor 30 may be configured to measure the temperature of the tissue 90 at more than one location, for example, more than one 3-D location within the tissue 90. In some embodiments, the temperature sensor is a magnetic-resonance thermometer. In some embodiments, the temperature sensor comprises one or more temperature-sensitive catheters (e.g., a thermistor disposed in a catheter).

In some embodiments, the system 10 may include a light-transmitting catheter 22 (e.g., a transparent catheter)(an “LTC”). In such embodiments, the optical fiber(s) 20 of the system 10 may be disposed within a corresponding number of LTCs 22. For example, each optical fiber 20 may be disposed in a lumen 23 of an LTC 22.

The system 10 further includes a controller 40 in communication with the temperature sensor 30. The controller 40 is configured to modify a fluence rate of the treatment light based on the temperature of the tissue 90, as described above. In some embodiments, the controller 40 is a programmable microprocessor, programmed to modify the treatment light based on a signal received from the temperature sensor. The treatment light may be modified by adjusting the intensity of the light source, attenuating the light emitted from the light source, independently attenuating the light in each optical fiber, or any other technique for increasing or decreasing the fluence rate of the treatment light delivered to the tissue via the optical fiber(s).

Some embodiments of the presently-disclosed system may include a dosimetry fiber 25 for measuring a light dose. In embodiments wherein the system includes an LTC 22, the dosimetry fiber 25 may be disposed in an LTC 22, for example, within a lumen 23 of an LTC 22. The dosimetry fiber 25 may be operably coupled to a spectrometer 27 for measuring the light dose delivered to the tissue. In this way, a desired total dose may be delivered while also maintaining the temperature of the tissue in a desired range for effective photothermal ablation (or other synergistic effect when combined with I-PDT).

The present disclosure is further illustrated in the discussion below, which includes exemplary embodiments used to test the disclosed technique using mouse models.

### Further Discussion

The mechanism of action in photodynamic therapy (PDT) involves the generation of reactive oxygen species and radicals through light activation of photosensitizer in the presence of oxygen—i.e., an effective photoreaction. Previous studies demonstrated that oxygen-conserving light fluence rate (mW/cm²) is required for an effective photoreaction. In I-PDT, multiple optical fibers with a cylindrical diffuser or optical fibers with flat cut end are inserted into the tumor. During I-PDT, the light dose rate (mW/cm) dictates the resulted fluence rate in the tumor. Thus, the light fluence rate depends on the light dose rate delivered from the optical fibers.

In the U.S., Photofrin is the only approved photosensitizer in the treatment of obstructing esophageal, non-small cell endobronchial lung cancer and high-grade dysplasia in Barrett's esophagus. The FDA-approved light dose rate for I-PDT with Photofrin is 400 mW/cm length of the optical fiber cylindrical diffuser, which translates to a fluence rate of up to 800 mW/cm². This clinically-approved light dose rate induces significant thermal ablation that could overwhelm the photoreaction. However, our preclinical data suggest that this is not the case, and cure of locally advanced squamous cell carcinoma can be achieved in a mouse model treated with I-PDT and thermal ablation. In an ongoing preclinical study it was found that the laser light can induce significant heating that can induce immediate tissue ablation, at T>60° C., without impeding the efficacy of the I-PDT.

The inventors hypothesized that the limited cure rate for I-PDT with porfimer sodium is due to the high dose rate that could limit the photodynamic efficiency by depleting tumor oxygen levels.

As further described below, aspects of the presently-disclosed method and system were implemented for testing. In particular, in vivo magnetic resonance thermometry (MRT) was used to quantify photothermal ablation during I-PDT. Time-to-event and cure rates were analyzed with Kaplan Meier curves. Safe and effective light dose rates and doses with photothermal ablation were identified.

### Animal Model

All procedures were carried out in accordance with a protocol approved by the Institutional Animal Care and Use Committee (IACUC) at Roswell Park Cancer Institute (RPCI). Female C3H, 8-12 weeks, mice bearing locally advanced SCCVII tumors were treated when the tumors reached a size of 9-10 mm in their largest diameter, and a volume of 400-500 mm³ calculated from caliper measurements, as is known in the art.

The animals were randomly assigned to receive light only (no drug) or I-PDT by administering 5 mg/kg porfimer sodium 24 hours prior to light delivery. Mice were considered cured if there was no palpable tumor any time at or after 60 days post I-PDT.

### Interstitial Photodynamic Therapy (I-PDT)

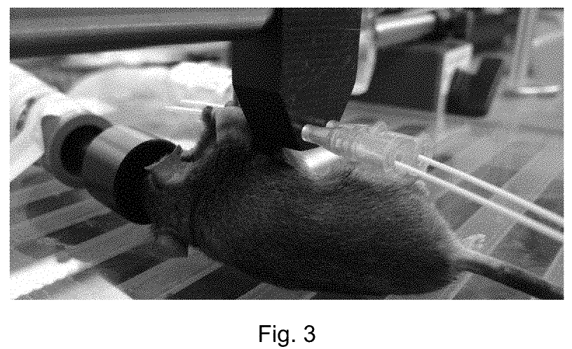

The laser light (treatment light) was delivered through one to three 0.98 mm diameter optical fibers with 20 mm cylindrical light diffuser RD20 (Medlight SA, Ecublens, Switzerland). The cylindrical light diffusers were connected to 1.0 Watt laser diode modules that emit 630±3 nm light (ML6500, Modulight Inc., Tampere, Finland). During light delivery, a custom made template was utilized to guide the placement of the laser fibers into the tumor, as shown in FIG. 3.

### Magnetic Resonance Thermometry

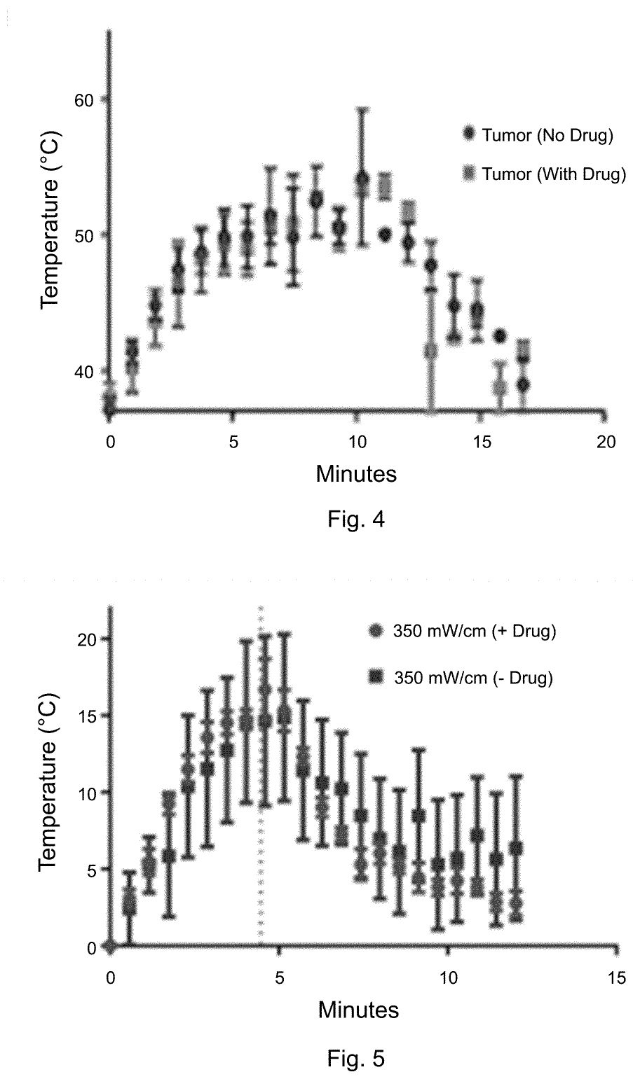

Magnetic resonance thermometry (MRT) by proton resonance frequency methods was carried out in a 4.7 Tesla preclinical scanner using the ParaVision 3.0.2 imaging platform (Bruker Biospin, Billerica MA) and a 35 mm quadrature transceiver coil. FIGS. 4 and 5 show that there was a temperature increase during the interstitial light delivery, with and without drug. These temperatures were averaged over the entire tumor volume.

### Results

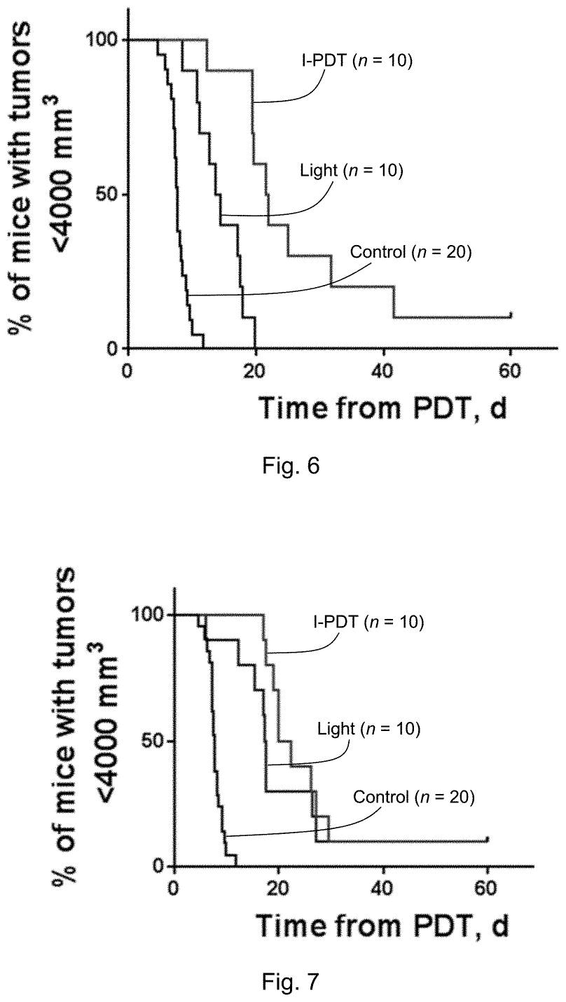

FIG. 6 shows the percentage of mice with tumors less than 4000 mm³ over a period of 60 days post treatment. As shown in the chart, a control population was untreated, a second population was treated with a light dose of 150 mW/cm, 100 J/cm (control vs. light p≤0.0001), and a third population was treated with I-PDT and similar dose (control vs. I-PDT p≤0.0001; light vs. I-PDT p=0.0004). FIG. 7 is a similar chart for populations treated with a light dose of 350 mW/cm, 100 J/cm (control vs. light p≤0.0001; control vs. I-PDT p≤0.0001; light vs. I-PDT p=0.339). It can be seen that after 60 days, only 10% of mice remained with tumors less than 4,000 mm³.

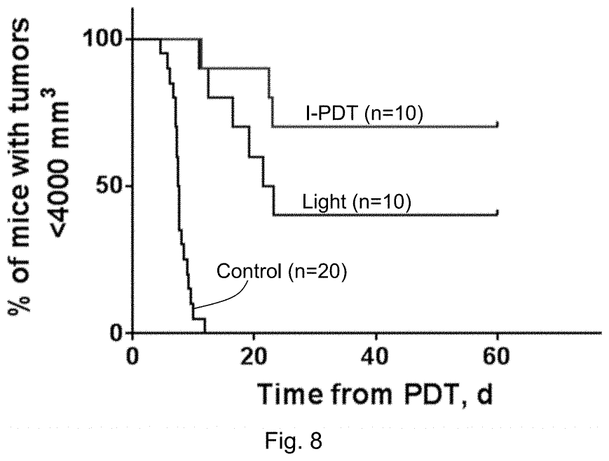

FIG. 8 shows the beneficial results of an embodiment of the presently-disclosed techniques using concurrent I-PDT and thermal ablation. It can be seen that the use of light without photosensitizer results in a cure rate of 40%. Despite the previous consideration that a lack of significant temperature change of the tissue was a benefit of PDT, FIG. 8 shows that concurrent photothermal ablation and I-PDT resulted in a cure rate of 70%.

Although claimed subject matter is described in terms of certain embodiments, other embodiments, including embodiments that do not provide all of the benefits and features set forth herein, are also within the scope of this disclosure. Various structural, logical, process step, and electronic changes may be made without departing from the scope of the disclosure.

What is claimed is:

1. A method for treating a tissue, comprising:
administering a photosensitizer to the tissue;
providing one or more optical fibers placed in the tissue;
applying a treatment light to the tissue by way of the one or more optical fibers, the treatment light having a fluence rate;
measuring a temperature of the tissue during application of the treatment light; and
modifying the fluence rate of the treatment light based on the temperature of the tissue.

2. The method of claim 1, wherein the fluence rate is modified to be lower if the temperature of the tissue is higher than a predetermined threshold.

3. The method of claim 1, wherein the fluence rate is modified to maintain a tissue temperature between 50° C. and 65° C.

4. The method of claim 1, wherein the fluence rate is modified to maintain a tissue temperature of substantially 60° C.

5. The method of claim 1, wherein the fluence rate is modified to maintain a tissue temperature between 60° C. and 90° C.

6. The method of claim 1, further comprising providing one or more dosimetry fibers placed in the tissue and configured to measure light dose.

7. The method of claim 6, wherein the optical fiber(s) and/or the dosimetry fiber(s) are disposed within one or more light-transmitting catheters (LTCs) placed in the tissue.
